(19) United States
(12) Patent Application Publication
(10) Pub. No.: US 2017/0258842 A1
(43) Pub. Date: Sep. 14, 2017

(54) Methods and Compositions for Treating Ovarian Failure

(71) Applicant: Augusta University Research Institute, Inc., Augusta, GA (US)

(72) Inventor: Ayman Al-Hendy, Augusta, GA (US)

(21) Appl. No.: 15/452,854

(22) Filed: Mar. 8, 2017

Related U.S. Application Data
(60) Provisional application No. 62/305,844, filed on Mar. 9, 2016.

(51) Int. Cl. A61K 35/28; A61K 9/00

(52) U.S. Cl. CPC A61K 35/28; A61K 9/0024

(57) ABSTRACT

Methods and compositions for treating ovarian failure are provided. In one embodiment, the method includes administering stem cells into the ovary of a female subject in need of such treatment. The stem cells are preferably bone marrow derived stem cells (BMSC). In other embodiments, the stem cells are embryonic stem cells, adult stem cells, induced stem cells, induced pluripotent stem cells, umbilical cord blood cells, or combinations thereof. The stem cells can be autologous or heterologous. In one embodiment, the stem cells have the following surface marker profile: CD105 positive, CD166 positive, CD90 positive, and CD73 positive as well as CD14 negative, CD34 negative, CD45 negative, HLA-DR negative, and CD 19 negative. The stem cells are administered in an amount effective to restore ovarian hormone production and promote folliculogenesis.

### CROSS-REFERENCE TO RELATED APPLICATIONS

This application claims priority to and benefit of U.S. Provisional Patent Application No. 62/305,844 filed on Mar. 9, 2016, and which is incorporated by reference in its entirety.

### FIELD OF THE INVENTION

The invention is generally directed to methods and compositions for treating ovarian infertility.

### BACKGROUND OF THE INVENTION

Premature ovarian failure (POF), also known as primary ovarian insufficiency, refers to a loss of normal ovarian function before age 40. In a normal karyotype female, POF is characterized by amenorrhea and infertility, with elevated serum levels of follicle-stimulating hormone (FSH) and decreased levels of estrogen. Currently, there is no effective treatment for this condition, and alternatives such as the use of egg donations are prohibitively expensive and ethically unacceptable to some couples. Xiao G Y, Liu I H, Cheng C C, Chang C C, Lee Y H, Cheng W T, et al. Amniotic fluid stem cells prevent follicle atresia and rescue fertility of mice with premature ovarian failure induced by chemotherapy. PloS one. 2014; 9(9):e106538.

Approximately 600,000 American women are diagnosed with cancer each year in the US, of whom 8% are of pre-reproductive age. Jemal A, Ward E, Hao Y, Thun M. Trends in the leading causes of death in the United States, 1970-2002. Jama. 2005; 294(10):1255-9. According to the Surveillance, Epidemiology, dand End Results (SEER) Cancer Statistics Review, 1975-2011, prevalence of cancer among females 20-29 years of age in the US is 88.223; 30-39 years of age is 226.162; and, 40-49 years of age is 638.879. Howlader N N A, Krapcho M, Garshell J, Miller D, Altekruse S F, Kosary C L, Yu M, Ruhl J, Tatalovich Z, Mariotto A, Lewis D R, Chen H S, Feuer E J, Cronin K A (eds). SEER Cancer Statistics Review, 1975-2011, National Cancer Institute. Bethesda, Md., http://seer.cancer.gov/csr/1975_2011/, based on November 2013 SEER data submission, posted to the SEER web site, April 2014. SEER Cancer Statistics Review, 1975-2011, National Cancer Institute. Bethesda, Md. 2014. Chemotherapy (CTX) is the most common treatment modality used in cancer patients followed by surgery, radiotherapy and other more specialized treatments. Lee H J, Selesniemi K, Niikura Y, Niikura T, Klein R, Dombkowski D M, et al. Bone marrow transplantation generates immature oocytes and rescues long-term fertility in a preclinical mouse model of chemotherapy-induced premature ovarian failure. Journal of clinical oncology: official journal of the American Society of Clinical Oncology. 2007; 25(22):3198-204. Premature ovarian failure (POF) is a common long-term consequence of CTX and radiotherapy. Both treatment modalities pose a significant risk of gonadal damage with severity of damage linked directly to dose and age. Anti-cancer therapeutic agents such as Cyclophosphamide and Busulfan are alkylating agents typically associated with gonadal damage. Averette H E, Boike G M, Jarrell M A. Effects of cancer chemotherapy on gonadal function and reproductive capacity. CA: a cancer journal for clinicians. 1990; 40(4):199-209. These chemotherapeutic agents have been shown to destroy ovarian follicles. Plowchalk D R, Mattison D R. Reproductive toxicity of cyclophosphamide in the C57BL/6N mouse: 1. Effects on ovarian structure and function. Reproductive toxicology (Elmsford, N.Y.). 1992; 6(5):411-21. Studies have shown that subsequent to CTX treatment, female mice demonstrated elevated levels of FSH, decreased estrogen levels, and sterility. These observations were attributed to the absence of folliculogenesis, and small non-functional ovaries. Abd-Allah S H, Shalaby S M, Pasha H F, El-Shal A S, Raafat N, Shabrawy S M, et al. Mechanistic action of mesenchymal stem cell injection in the treatment of chemically induced ovarian failure in rabbits. Cytotherapy. 2013; 15(1):64-75. Accordingly, this CTX-induced ovarian failure model is currently the most reliable, and thereby the most commonly used preclinical model for the evaluation and study of ovarian failure in mice.

The American Society of Clinical Oncology and the American Society for Reproductive Medicine have issued guidelines stipulating the potential adverse effect of cancer treatment on fertility, and recommend that fertility preservation options be presented to patients in the initial stages of treatment. Abd-Allah S H, Shalaby S M, Pasha H F, El-Shal A S, Raafat N, Shabrawy S M, et al. Mechanistic action of mesenchymal stem cell injection in the treatment of chemically induced ovarian failure in rabbits. Cytotherapy. 2013; 15(1):64-75. Existing approaches aimed at preserving fertility in female cancer survivors include: protection of ovarian follicles with gonadotropin releasing hormone agonist (GnRH-a) at time of administering the CTX and/or ovarian tissue, ova or embryo freezing prior to CTX, followed by tissue re-implantation or utilization of assisted reproductive techniques. Fertility preservation and reproduction in patients facing gonadotoxic therapies: a committee opinion. Fertility and sterility. 2013; 100(5):1224-31. Regretfully, these procedures have several important limitations such as; inconsistent outcomes or need for invasive surgical procedures to permit recovery of ovarian tissue or prolonged ovulation induction accompanied by ultrasound guided egg retrieval and subsequent assisted reproductive technology (ART). For many patients, this is cost prohibitive and can be potentially deleterious to their reproductive capacity. Furthermore, the latter procedures are not considered to be viable options for young prepubertal girls. Additionally, some of the advanced technology required for these options are not readily available, globally. Clearly, less invasive and more cost-effective approaches are imperative for the preservation of fertility in prepubertal and reproductive age female cancer survivors.

Currently, controversy centers around which specific cell type(s) within the ovarian follicles are affected by CTX. Desmeules P, Devine P J. Characterizing the ovotoxicity of cyclophosphamide metabolites on cultured mouse ovaries. Toxicological sciences: an official journal of the Society of Toxicology. 2006; 90(2):500-9. Some studies report that chemotherapeutic agents appear to directly target oocytes in primordial and primary follicles for apoptotic destruction. Desmeules P, Devine P J. Characterizing the ovotoxicity of cyclophosphamide metabolites on cultured mouse ovaries. Toxicological sciences: an official journal of the Society of Toxicology. 2006; 90(2):500-9. However, this is in stark contrast to evidence which shows that rapidly dividing cells are more sensitive to the cytotoxic effects of alkylating agents than are cells at rest making steroidogenic cells of the follicle, the likely target for CTX. Blumenfeld Z. Gynaecologic concerns for young women exposed to gonadotoxic chemotherapy. Current opinion in obstetrics & gynecology. 2003; 15(5):359-70. Therefore, it is highly unlikely that postnatal oocytes which are at the dictyate state of arrest and do not undergo mitosis are susceptible to apoptotic destruction by alkylating agents used for cancer CTX. Blumenfeld Z. Gynaecologic concerns for young women exposed to gonadotoxic chemotherapy. Current opinion in obstetrics & gynecology. 2003; 15(5):359-70. Blumenfeld Z, Avivi I, Ritter M, Rowe J M. Preservation of fertility and ovarian function and minimizing chemotherapy-induced gonadotoxicity in young women. Journal of the Society for Gynecologic Investigation. 1999; 6(5):229-39.

Human BMSCs transplantation has been cited in more than 344 clinical trials for the treatment of a variety of diseases all over the world. Many studies indicate that when stem cells are transplanted into a specific microenvironment, they are stimulated by the niche, and while the release of cell growth factors stimulate the surrounding tissue regeneration, the cells may also be induced to differentiate into specific tissue or organ-like cells. Abd-Allah S H, Shalaby S M, Pasha H F, El-Shal A S, Raafat N, Shabrawy S M, et al. Mechanistic action of mesenchymal stem cell injection in the treatment of chemically induced ovarian failure in rabbits. Cytotherapy. 2013; 15(1):64-75. Wei X, Yang X, Han Z P, Qu F F, Shao L, Shi Y F. Mesenchymal stem cells: a new trend for cell therapy. Acta pharmacologica Sinica. 2013; 34(6):747-54. Fu X, He Y, Xie C, Liu W. Bone marrow mesenchymal stem cell transplantation improves ovarian function and structure in rats with chemotherapy-induced ovarian damage. Cytotherapy. 2008; 10(4):353-63. Moreover, mesenchymal stem cells (MSCs) can be used in allogeneic settings without immunosuppressive therapy due to their ability to evade immune detection (immune-privileged status, although the exact mechanism is not entirely understood). Kimbrel E A, Kouris N A, Yavanian G J, Chu J, Qin Y, Chan A, et al. Mesenchymal stem cell population derived from human pluripotent stem cells displays potent immunomodulatory and therapeutic properties. Stem cells and development. 2014; 23(14):1611-24. The many advantages of the mesenchymal approach include: accessibility, versatility and a thoroughly vetted review of the literature with respect to the various health benefits. Nevertheless, stem cell transplantation in the treatment of human diseases must be safe and effective. Stem cells transplanted into the host could not only induce immune rejection by the host but also result in cancerous tissue or distortions in the host body. With that said, great interest has developed in MSCs, which are free of both ethical concerns and teratoma formation as compared to embryonic stem cells and induced pluripotent stem cells. Wei X, Yang X, Han Z P, Qu F F, Shao L, Shi Y F. Mesenchymal stem cells: a new trend for cell therapy. Acta pharmacologica Sinica. 2013; 34(6):747-54.

It has been shown that a variety of diseases, including reproductive dysfunction, can be treated by allogeneic bone marrow stem cells (BMSCs). Lee H J, Selesniemi K, Niikura Y, Niikura T, Klein R, Dombkowski D M, et al. Bone marrow transplantation generates immature oocytes and rescues long-term fertility in a preclinical mouse model of chemotherapy-induced premature ovarian failure. Journal of clinical oncology: official journal of the American Society of Clinical Oncology. 2007; 25(22):3198-204. Selesniemi K, Lee H J, Niikura T, Tilly J L. Young adult donor bone marrow infusions into female mice postpone age-related reproductive failure and improve offspring survival. Aging. 2009; 1(1):49-57.

Based on gene expression analyses and bone marrow transplantation (BMT) experiments using CTX sterilized recipients, Johnson et al. 2007 showed that in adult female mice, BMT rescued long-term fertility in CTX-treated females, but all offspring were derived from the recipient germ cells. Lee H J, Selesniemi K, Niikura Y, Niikura T, Klein R, Dombkowski D M, et al. Bone marrow transplantation generates immature oocytes and rescues long-term fertility in a preclinical mouse model of chemotherapy-induced premature ovarian failure. Journal of clinical oncology: official journal of the American Society of Clinical Oncology. 2007; 25(22):3198-204. This suggests that BMT rescues long-term fertility by either protecting existing oocytes from CTX or reinstituting recipient oogenesis.

Thus, there is a need for methods and compositions for restoring ovarian hormone production and for promoting folliculogenesis.

### SUMMARY OF THE INVENTION

Methods and compositions for treating ovarian failure are provided. In one embodiment, the method includes administering stem cells, preferably human, into the ovary of a female subject, preferably a human female. The stem cells are preferably human bone marrow mesenchymal stem cells (BMSC). In other embodiments, the stem cells are embryonic stem cells, adult stem cells, induced stem cells, induced pluripotent stem cells, umbilical cord blood cells, or combinations thereof. The stem cells can be autologous or heterologous. In some embodiments, the stem cells are collected from the subject to be treated and are expanded in cell culture prior to administration to the subject. In one embodiment, the stem cells selected to have the following surface marker profile: CD105 positive, CD166 positive, CD90 positive, and CD73 positive as well as CD14 negative, CD34 negative, CD45 negative, HLA-DR negative, and CD 19 negative. The selected stem cells are administered or grafted directly into or on the ovary in an amount effective to restore ovarian hormone production and promote folliculogenesis

The ovarian failure to be treated includes idiopathic premature ovarian failure as well as chemotherapy-induced ovarian failure. The stem cells can be administered to the subject prior to chemotherapy treatment.

Still another embodiment provides a method for treating ovarian failure in a subject in need thereof by administering platelet enriched plasma into an ovary of the subject in an amount effective to restore ovarian hormone production and promote folliculogenesis.

Another embodiment provides a method for treating ovarian failure in a subject in need thereof by locally administering to an ovary of the subject an effective amount of conditioned cell culture medium harvested from cultured stem cells, wherein the cultured stem cells are CD105 positive, CD166 positive, CD90 positive, and CD73 positive as well as CD14 negative, CD34 negative, CD45 negative, HLA-DR negative, and CD 19 negative.

### BRIEF DESCRIPTION OF THE DRAWINGS

FIG. 1 is a graph of the mean cycle length (days) by Group: Group 1 and 3 differed significantly in the mean number of days in the diestrous cycle and the mean number of days for the total cycle than group 2 (p-value <0.0001).

FIG. 2 is a line graph of Mean Body weight (grams) versus Group and Time (weeks). Group 2, the trend was such that body weight remained relatively constant over time post-treatment. Group 3, significant increases in body weight of 1.99 g from 2 to 6 weeks (p-value <0.0001), 2.75 g from 2 to 8 weeks (p-value <0.0001), 1.37 g, from 4 to 6 weeks (p-value=0.0005), and 2.13 g from 4 to 8 weeks (p-value <0.0001). At 6 and 8 week time points group 3 had significantly higher mean body weight than group 2 (p-value <0.0001).

FIG. 3A is a bar graph of Mean Left Ovary, Right Ovary, and Total (Left+Right) Ovary Weight (grams) by Group. Group 3 had significantly higher mean total ovary weight than group 2 by 6.09 g (p-value=0.0022); there was not a significant difference in mean total ovary weight between group 1 and 3 (p-value=0.92). FIG. 3B is a bar graph of Mean Uterus and Vagina Weight (g) by Group. Group 1 had significantly greater mean uterus weight than group 2 by 74.30 g (p-value=0.0004) and significantly greater mean uterus weight than group 3 by 44.83 g (p-value=0.0158); there was not a significant difference in mean uterus weight between groups 2 & 3 (p-value=0.09). Group 1 had significantly greater mean vagina weight than group 2 by 8.92 g (p value <0.0001) and significantly greater mean vagina weight than group 3 by 8.62 g (p-value <0.0001); there was not a significant difference in mean vagina weight between groups 2 & 3 (p value=0.66). FIG. 3C is a bar graph of Mean Kidney, spleen, heart and Lung Weight (g) by Group. Group 1 had significantly greater mean kidney weight than group 2 by 133.40 g (p-value <0.0001) and significantly greater mean kidney weight than group 3 by 105.73 g (p-value=0.0005); there was not a significant difference in mean kidney weight between groups 2 & 3 (pvalue=0.27). 3c2. Group 2 had significantly lower mean heart weight than group 1 by 41.98 g (p-value=0.0025) and lower mean heart weight than group 3 by 28.20 g (p-value=0.0281), however the difference with group 3 did not remain significant after adjusting for multiple comparisons; there was not a significant difference in mean heart weight between groups 1 & 3 (p-value=0.25). FIG. 3D is a bar graph of Mean Liver Weight (g) by Group. Group 3 had significantly higher mean liver weight as than group 2 by 264.98 g (p-value=0.0006); there was not a significant difference in liver weight between groups 1 & 3 (p-value=0.31).

FIG. 4A is a bar graph of Mean FSH levels (ng/ml) by Group. At 4,6 & 8 weeks post treatment group 3 had significantly lower FSH than group 2 (p-value=0.0338, 0.0165, 0.0034) respectively. Groups 1 & 3 did not have significantly different FSH at 4, 6, 8 week time points (p-value >0.05) FIG. 4B is a bar graph of Mean Serum Estradiol (pg/ml) over Time (weeks) by Group. Group 3 had higher mean Serum Estradiol than group2 at 4 weeks (p-value=0.0066) and at 6 weeks (p-value=0.0064). FIG. 4C is a bar graph of Mean AMH (ng/ml) over Time (weeks) by Group. At 4 week time point group 1 had significantly higher AMH than the group (p-value <0.0001) and group 3 (p-value=0.0005). At 8 weeks post-treatment, and group 3 had greater AMH than group 2 by 42.11 (p-value=0.0042) and the groups 1 and 3 did not have significantly different AMH levels (p-value=0.9287). Within group 2 there were no significant differences in AMH over time (all p-values >0.05).

FIGS. 5A, 5B and 5C are micrographs showing Hematoxlyin and Eosin staining of the ovarian tissues 10×. FIG. 5A is of Group1 representing the normal ovarian tissue. FIG. 5B is of Group 2 had an overall reduction in ovary size and failed to develop antral follicles. FIG. 5C is of Group 3 showing the trend of increase in the number of follicles. FIG. 5D is a bar graph of Mean follicle count by group. The mean follicle count differs among the groups (p<0.0001). The mean follicle count in-group 1 was 5.4 times larger than group 2 and 1.8 times larger than group 3. The mean follicle count in group 3 was 1.92 times greater than group 2, however this difference was not statistically significant. FIG. 5E is a bar graph of Immunohistochemistry quantification data. The ANOVA models indicated that there were significant differences between the groups in the mean FSHR (p-value <0.0001), AMH (p-value <0.0001), Inhibin A (p-value <0.0001), and InhibinB (p-value=0.0004).

FIGS. 6A and 6B are bar graphs of Mean number of pregnancies and Mean number of pups per group, respectfully. There was not a significant difference in the mean number of pregnancies (p-value=0.0693), but a larger sample is likely to show significant differences. Pairwise comparisons suggest that the mean number of pregnancies is 12 times greater for group 1 than group 2 (p-value=0.0223) and 11.2 times greater for the group 3 than group 2(p-value=0.0263); groups 1 and 3 did not have different mean number of pregnancies (p-value=0.7867).

### DETAILED DESCRIPTION OF THE INVENTION

### I. Definitions

The use of the terms “a,” “an,” “the,” and similar referents in the context of describing the presently claimed invention (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

### II. Methods for Treating Ovarian Failure

The data provided herein show that implantation of human BMSCs into ovaries successfully treats ovarian failure and is minimally invasive, cost affordable, and widely applicable, especially for young patients after CTX treatment.

The chemotherapy-induced ovarian failure mouse model used in this work, is biologically representative of human POF as female mice when injected with CTX consistently have elevated serum levels of FSH, decreased E2 levels and are virtually reproductively sterile. The data show that mesenchymal stem cells extracted from the human bone marrow of a normal young female did efficiently graft into a preclinical chemotherapy induced ovarian failure immune-competent mouse model (CTX POF); stimulated follicular maturation to the antral stage; and, increased E2 & AMH production as well as decreased FSH secretion. These hormonal changes were consistently reflected in the reproductive physiology of this animal model.

Treated animals showed estrogenic changes in daily vaginal smears as early as 24 h after mesenchymal stem cell implantation while vaginal smears in control animals remained unchanged. Reproductive organs which are E2-responsive, showed a remarkable increase in weight at all-time points of the experiment. A trend of increased maturation and total number of the follicles in treated animals was also observed at 2, 4, 6 and 8 week timepoints post BMSCs implantation. Most importantly, there was observed ‘rescue’ of the infertility phenotype in treated mice versus untreated group.

Stem cells have emerged as a key element of regenerative medicine therapies due to their inherent ability to differentiate into a variety of cell phenotypes, thereby providing innumerable possibilities as cell therapy to treat degenerative diseases as well as their ability to secrete several bioactive factors that modulate surrounding cells. Wang S, Yu L, Sun M, Mu S, Wang C, Wang D, et al. The therapeutic potential of umbilical cord mesenchymal stem cells in mice premature ovarian failure. BioMed research international. 2013; 2013:690491.

Several investigators have demonstrated the mechanism (s) and robust regenerative abilities of BMSCs and other types of MSCs (such as Adipose derived, menstrual blood derived, skin derived and umbilical cord derived MSCs). Abd-Allah S H, Shalaby S M, Pasha H F, El-Shal A S, Raafat N, Shabrawy S M, et al. Mechanistic action of mesenchymal stem cell injection in the treatment of chemically induced ovarian failure in rabbits. Cytotherapy. 2013; 15(1):64-75. Fu X, He Y, Xie C, Liu W. Bone marrow mesenchymal stem cell transplantation improves ovarian function and structure in rats with chemotherapy-induced ovarian damage. Cytotherapy. 2008; 10(4):353-63. Takehara Y, Yabuuchi A, Ezoe K, Kuroda T, Yamadera R, Sano C, et al. The restorative effects of adipose-derived mesenchymal stem cells on damaged ovarian function. Laboratory investigation; a journal of technical methods and pathology. 2013; 93(2):181-93. Kilic S, Pinarli F, Ozogul C, Tasdemir N, Naz Sarac G, Delibasi T. Protection from cyclophosphamide-induced ovarian damage with bone marrow-derived mesenchymal stem cells during puberty. Gynecological endocrinology: the official journal of the International Society of Gynecological Endocrinology. 2014; 30(2):135-40; Cohn S L. Secreted protein acidic and rich in cysteine is a matrix scavenger chaperone. Pediatric blood & cancer. 2011; 6(9):e23880. The mechanisms by which MSCs exert their immunomodulatory and reparative effects are still largely unknown and may be linked to multiple mechanisms mediated by both soluble factors and cell contact. In addition, it is possible that interactions between immune cells and MSCs in the tissue microenvironment might modulate MSCs function and contribute to outcomes of MSCs-based therapy. Cipriani P, Ruscitti P, Di Benedetto P, Carubbi F, Liakouli V, Berardicurti O, et al. Mesenchymal stromal cells and rheumatic diseases: new tools from pathogenesis to regenerative therapies. Cytotherapy. 2015. Previous research has demonstrated that follicular atresia is predominantly mediated by the apoptosis of follicular cells, especially granulosa cells which are necessary for follicular development Hughes F M, Jr., Gorospe W C. Biochemical identification of apoptosis (programmed cell death) in granulosa cells: evidence for a potential mechanism underlying follicular atresia. Endocrinology. 1991; 129(5):2415-22; Kaipia A, Hsueh A J. Regulation of ovarian follicle atresia. Annual review of physiology. 1997; 59:349-63. Jiang J Y, Cheung C K, Wang Y, Tsang B K. Regulation of cell death and cell survival gene expression during ovarian follicular development and atresia. Frontiers in bioscience: a journal and virtual library. 2003; 8:d222-37. Nonetheless, no evidence indicating that implanted BMSCs directly differentiated into granulosa cells to replace damaged cells within the ovary of POF mice was found. It has been hypothesized that the restorative effects of MSCs transplantation on impaired ovarian function primarily results from the secretion of trophic factors, which are beneficial to follicular growth. Fu X, He Y, Xie C, Liu W. Bone marrow mesenchymal stem cell transplantation improves ovarian function and structure in rats with chemotherapy-induced ovarian damage. Cytotherapy. 2008; 10(4):353-63. Takehara Y, Yabuuchi A, Ezoe K, Kuroda T, Yamadera R, Sano C, et al. The restorative effects of adipose-derived mesenchymal stem cells on damaged ovarian function. Laboratory investigation; a journal of technical methods and pathology. 2013; 93(2):181-93. The data has shown higher FSHR staining in group 3 vs. group 2, suggesting that there is an increase in the number of granulosa cells, as these are the only cells in the female ovary that express FSHR. Either the BMSCs did actually differentiate into new granulosa cells or secreted trophic factors from BMSCs did revive the partially damaged endogenous mouse granulosa cells. Vimentin staining (indicative of the existence of human BMSCs) showed a circular distribution of such cells around the growing follicle in the ovarian stroma, seemingly in support of the latter option.

Previous studies demonstrated that BM mesenchymal stem cells can restore ovarian function and reactivate dormant folliculogenesis in several mouse models of ovarian dysfunction. Lee H J, Selesniemi K, Niikura Y, Niikura T, Klein R, Dombkowski D M, et al. Bone marrow transplantation generates immature oocytes and rescues long-term fertility in a preclinical mouse model of chemotherapy-induced premature ovarian failure. Journal of clinical oncology: official journal of the American Society of Clinical Oncology. 2007; 25(22):3198-204. Ghadami M, El-Demerdash E, Salama S A, Binhazim A A, Archibong A E, Chen X, et al. Toward gene therapy of premature ovarian failure: intraovarian injection of adenovirus expressing human FSH receptor restores folliculogenesis in FSHR(−/−) FORKO mice. Molecular human reproduction. 2010; 16(4):241-50. It was previously shown that bone marrow nucleated cells intravenously delivered into the FSH receptor (FSHR) knocked out (FORKO) female mice restored ovarian steroidogenesis. Ghadami M, El-Demerdash E, Salama S A, Binhazim A A, Archibong A E, Chen X, et al. Toward gene therapy of premature ovarian failure: intraovarian injection of adenovirus expressing human FSH receptor restores folliculogenesis in FSHR(−/−) FORKO mice. Molecular human reproduction. 2010; 16(4):241-50. Specifically in that work, the data demonstrated that this treatment restored FSHR expression in the ovaries, highly suggesting that implanted cells did in fact transdifferentiate into a granulosa-cell like structures. Ghadami M, El-Demerdash E, Salama S A, Binhazim A A, Archibong A E, Chen X, et al. Toward gene therapy of premature ovarian failure: intraovarian injection of adenovirus expressing human FSH receptor restores folliculogenesis in FSHR(−/−) FORKO mice. Molecular human reproduction. 2010; 16(4):241-50. It has also been demonstrated that BMSCs can contribute to the recovery of ovarian structure and function, injured by cyclophosphamide. Abd-Allah S H, Shalaby S M, Pasha H F, El-Shal A S, Raafat N, Shabrawy S M, et al. Mechanistic action of mesenchymal stem cell injection in the treatment of chemically induced ovarian failure in rabbits. Cytotherapy. 2013; 15(1):64-75. Kilic S, Pinarli F, Ozogul C, Tasdemir N, Naz Sarac G, Delibasi T. Protection from cyclophosphamide-induced ovarian damage with bone marrow-derived mesenchymal stem cells during puberty. Gynecological endocrinology: the official journal of the International Society of Gynecological Endocrinology. 2014; 30(2):135-40. During the course of this study, human BMSCs, derived from the human bone marrow of a 21 year-old white female, were implanted into the ovaries of a POF mice model suggesting that stem cells from a human source can act as a viable xenogeneic transplant donor; hence, it is not a far reaching concept that they will likely act as well or better in the CTX-damaged allogeneic human ovary considering their immune-privileged status. Kimbrel E A, Kouris N A, Yavanian G J, Chu J, Qin Y, Chan A, et al. Mesenchymal stem cell population derived from human pluripotent stem cells displays potent immunomodulatory and therapeutic properties. Stem cells and development. 2014; 23(14):1611-24. Previous studies have shown an effect of donor's age on the proliferation capacity of mesenchymal stem cells isolated from BM which correlated with a decrease in clonogenicity. Li Y, Charif N, Mainard D, Bensoussan D, Stoltz J-F, de Isla N. Donor's age dependent proliferation decrease of human bone marrow mesenchymal stem cells is linked to diminished clonogenicity. Bio-Medical Materials And Engineering. 2014; 24(1 Suppl):47-52.

Cancer CTX using busulfan/cyclophosphamide was designed to ablate highly proliferating cells by inducing the cross-linking of DNA Helleday T, Petermann E, Lundin C, Hodgson B, Sharma R A. DNA repair pathways as targets for cancer therapy. Nature reviews Cancer. 2008; 8(3):193-204, which affects the mitochondria and results in the activation of the apoptotic pathway. Zhao X J, Huang Y H, Yu Y C, Xin X Y. GnRH antagonist cetrorelix inhibits mitochondria-dependent apoptosis triggered by chemotherapy in granulosa cells of rats. Gynecologic oncology. 2010; 118(1):69-75. Accordingly, it is widely believed that chemotherapeutic drugs may massively eliminate granulosa cells Desmeules P, Devine P J. Characterizing the ovotoxicity of cyclophosphamide metabolites on cultured mouse ovaries. Toxicological sciences: an official journal of the Society of Toxicology. 2006; 90(2):500-9, which are required for oocyte survival and follicular development. Matzuk M M, Burns K H, Viveiros M M, Eppig J J. Intercellular communication in the mammalian ovary: oocytes carry the conversation. Science (New York, N.Y.). 2002; 296(5576):2178-80. The ensuing damage to growing follicles by CTX increases recruitment of primordial follicles and, in turn, the total number of all types of follicles is eventually reduced. Rosendahl M, Andersen C Y, la Cour Freiesleben N, Juul A, Lossl K, Andersen A N. Dynamics and mechanisms of chemotherapy-induced ovarian follicular depletion in women of fertile age. Fertility and sterility. 2010; 94(1):156-66. The follicle consists of germ cells that become ova, granulosa and theca cells, which produce steroid hormones. Folliculogenesis requires a carefully orchestrated cross talk between germ cells and surrounding somatic cells, however, CTX could destroy these ovarian niche interactions, decrease granulosa cell function, and ultimately induce ovarian toxicity and function failure. Sklar C. Reproductive physiology and treatment-related loss of sex hormone production. Medical and pediatric oncology. 1999; 33(1):2-8. Follicle counts conducted after CTX treatment reveal vastly reduced primordial, primary and secondary follicles, whereas BMSCs implantation in the ovaries reconstituted the follicle reserve. No evidence exists to suggest that BMSCs can differentiate into oocytes in recipient mice. Herein, the number of primordial and growing follicles after BMSCs implantation were not equivalent to the numbers found in normal female mice of the same age. This observation may explain why BMSCs implantation did not completely restore fertility in CTX-induced sterilized mice to normal levels (normal mice in group 1, not exposed to CTX).

Taken together, the favorable effects of BMSCs on CTX damaged ovaries is derived primarily from the paracrine secretion of certain beneficial secreted growth factors, which support ovarian follicle survival. Since ovarian granulosa cells are essential to sustain follicle survival, it is likely that the interaction between BMSCs and granulosa cells plays a key role in this favorable effect.

In one embodiment, multiple BMSCs injections are administered closer to initiation or even prior to CTX in an effort to better: 1) preserve follicle reserve from destruction, 2) prevent POF from occurring; and, 3) to ensure maximal efficacy is achieved. The proliferative capacity of MSCs in vitro is limited, and its acquisition is partially invasive; Baxter M A, Wynn R F, Jowitt S N, Wraith J E, Fairbairn L J, Bellantuono I. Study of telomere length reveals rapid aging of human marrow stromal cells following in vitro expansion. Stem cells (Dayton, Ohio). 2004; 22(5):675-82; therefore, an alternative cell source could be considered for the clinical application of BMSCs in the treatment of POF. Other cells that can be used include, but are not limited to umbilical cord blood mesenchymal stem cells.

Another embodiment provides a method for treating ovarian failure in a subject in need thereof by administering an effective amount of bone marrow derived stem cells into an ovary of the subject to restore ovarian hormone production and promote folliculogenesis. The ovarian failure can be idiopathic premature ovarian failure or chemotherapy-induced ovarian failure.

In one embodiment the bone marrow derived stem cells have the following surface marker profile: CD105 positive, CD166 positive, CD90 positive, and CD73 positive as well as CD14 negative, CD34 negative, CD45 negative, HLA-DR negative, and CD 19 negative.

The bone marrow derived stem cells are preferably autologous human stem cells. In certain embodiments the bone marrow derived stem cells are expanded in cell culture prior to administration to the subject. In other embodiments the ovarian cells are heterologous.

Another embodiment provides a method for treating chemotherapy-induced ovarian failure in a subject in need thereof by administering locally to an ovary of the subject an effective amount of stem cells to restore ovarian hormone production and promote folliculogenesis. The stem cells can be selected from the group consisting of embryonic stem cells, adult stem cells, induced pluripotent stem cells, umbilical cord blood cells, placental blood cells, hematopoietic stem cells, and combinations thereof. The stem cells are preferably CD105 positive, CD166 positive, CD90 positive, and CD73 positive as well as CD14 negative, CD34 negative, CD45 negative, HLA-DR negative, and CD 19 negative.

Another embodiment provides a method for treating ovarian failure in a subject in need thereof by administering platelet enriched plasma directly into an ovary of the subject in an amount effective to restore ovarian hormone production and promote folliculogenesis.

Still another embodiment provides a method for treating ovarian failure in a subject in need thereof by locally administering to an ovary of the subject an effective amount of conditioned cell culture medium harvested from cultured stem cells, wherein the cultured stem cells are CD105 positive, CD166 positive, CD90 positive, and CD73 positive as well as CD14 negative, CD34 negative, CD45 negative, HLA-DR negative, and CD 19 negative.

### Examples

### Example 1: Vaginal Smear Changes

Material and Methods

Animals

Adult female C57BL6 mice, 4-6 weeks of age, weighing between 20-25 g, were purchased from Charles River Co. (Wilmington, Mass.). Animals were housed in groups of six within polyethylene cages and allowed to acclimatize to the animal facility environment for at least one week prior to study initiation. Animals were maintained in an environmentally controlled room with 12 hour light: 12 hour dark cycles (lights on at 6:00 a.m.), 22° C. with a humidity range of 50% to 60%. Animals were allowed to access commercial pelleted mouse chow and water.

All animal procedures were approved by Georgia Regents University's (GRU) Institutional Animal Care and Use Committee (IACUC), and performed in accordance with the National Research Council Guide for Care and Use of Laboratory Animals. The protocol was approved by the GRU Institutional Review Board (IRB). All surgeries were performed under Isoflurane inhalation anesthesia, and mice were placed under a heating red lamp to maintain body temperature until recovery.

Treatment Experiment Acclimatized mice (N=6/group) were randomly assigned to: a control group (group1); a sham chemotherapy group (group 2); or, a stem cell-based treatment group (group 3). Chemotherapy treatment consisted of a single IP injection of Busulfan (12 mg/kg) and Cyclophosphamide (70 mg/kg), both dissolved in saline. This combination is hereafter referred to as CTX. Seven days post injection, mice in groups 1 and 2 were subjected to bilateral intra-ovarian implantation of 10 μl phosphate buffered saline via abdominal laparotomy. Group 3 received 10 μl of BMSCs suspension (5×10⁵ cells) in each ovary.

Preparation of Stem Cells

Stem cells were collected using the following method. Samples of human bone marrow aspirates were collected from women undergoing diagnostic bone marrow aspiration for clinical indications. These women had not used any hormonal treatment for at least 3 months before the day of their procedure (day of sample collection). The menstrual phase was consistently captured for all the tissue collection, according to subject history and subsequently validated by endometrial histology. The samples were collected in the proliferative phase of the menstrual cycle. Use of human tissue specimens was approved by the Institutional Review Board and Ethics Committee, and all patients signed a written informed consent form. Consistently, collected aspirate were rinsed in wash buffer solution containing Hank's Balanced Salt Solution (Life Technologies) and 1% antibiotic-antimycotic solution (Life Technologies). Samples were carefully manually minced into small aggregates (<1 mm3) and further dissociated using the gentle MACS dissociator (Milteny Biotec). Then they were suspended in enzyme buffer containing collagenase IV and DNAse I and digested overnight at 37° C. by enzymatic means. Magnetic bead selection was subsequently performed according to the manufacturer's instructions (Life Technologies). Freshly isolated cell suspensions were incubated with biotinylated and conjugated antibodies to specific surface markers (CD105, BD Biosciences, and CD166, R&D Systems), diluted in isolation buffer containing phosphate-buffered saline (Sigma-Aldrich) and supplemented with 0.1% bovine serum albumin (Sigma-Aldrich) and 2 mM of ethylene diamine tetraacetic acid. Dynabeads FlowComp (Life Technologies) were then added, and tubes containing cell suspensions were placed in a magnet to separate the candidate CD105/CD166-positive cells from the supernatant containing nontarget cells, repeating this step in triplicate to remove any residual beads. Finally, CD105/CD166 human bone marrow mesenchymal stem cells were cultured in Dulbecco's modified Eagle's medium (Sigma-Aldrich) with 10% fetal bovine serum (Stem Cell Technologies) and 1% antibiotic-antimycotic (Life Technologies), and plated in collagen coated dishes. These CD105/CD166 positive cultured human bone marrow mesenchymal stem cells were dissociated from the collagen-coated dishes by using non-enzymatic cell dissociation buffer (Gibco). They were washed and resuspended in cold stain buffer, to perform the immunophenotypic analysis using various surface markers. Similarly, isotype controls were prepared for each antibody in separate aliquots. Using flow cytometric (FACS) analysis, it was found that the surface marker profile of these cells is as follows: CD105 positive, CD166 positive, CD90 positive, and CD73 positive as well as CD14 negative, CD34 negative, CD45 negative, HLA-DR negative, and CD 19 negative.

Ovarian Implantation of BMSCs

BMSCs were purchased from LONZA INC., USA (catalogue number PT-2501) and grown in accordance to the manufacturer's manual or prepared as described above. Cells were plated in vitro per the manufacturer's instructions up to passage 3 and suspended in 10 μl mesenchymal stem media from Stem Cell Technology, Vancouver, Canada (catalogue number 05401). Cells were injected into both ovaries (right and left) of each animal in group 3 at a concentration of 5×10⁵ cells per ovary.

Daily Vaginal Smear and Total Body Weight

Mice in all groups were weighed weekly and subjected to daily vaginal swabs for 21 days to determine CTX's effect on stages and overall length of the estrous cycle. Characteristic features of specific phases, as described by Rugh R, Ginns E I, Ho H S, Leach W M. Responses of the mouse to microwave radiation during estrous cycle and pregnancy. Radiation research. 1975; 62(2):225-41., were as follows: a smear consisting almost exclusively of leukocytes depicted diestrus; a thin smear of equal numbers of leukocytes and elongated nucleated epithelium indicated proestrus; large cornified epithelial cells were exclusively found in estrus; and, metestrus was marked by a thick smear composed of equal numbers of nucleated epithelial cells and leukocytes. Mice entered the estrus stage in the early hours of the night. Circulating levels of Estradiol (E2) peaked prior to ovulation, which occurs at estrus, while Progesterone (P4) levels rise during metestrus and diestrus, and then declined from proestrus to estrus. Walmer D K, Wrona M A, Hughes C L, Nelson K G. Lactoferrin expression in the mouse reproductive tract during the natural estrous cycle: correlation with circulating estradiol and progesterone. Endocrinology. 1992; 131(3):1458-66. Fata J E, Chaudhary V, Khokha R. Cellular turnover in the mammary gland is correlated with systemic levels of progesterone and not 17beta-estradiol during the estrous cycle. Biology of reproduction. 2001; 65(3):680-8.

Experiment Timeline:

Timepoints were set at 2, 4, 6 and 8 weeks from day of surgery; At each timepoint animals were weighed, blood samples were collected and hormonal assays were conducted. An animal from each group was sacrificed for the harvesting of tissue samples for further studies. The left-side ovary was excised from each animal (N=6/group) and fixed in 4% buffered formalin for 24 hours prior to storage (in 70% ethanol) until paraffin blocked and sectioned. Ovarian tissue sections (5 μm thick) were subsequently subjected to hematoxylin and eosin (H&E) to assess the distribution of ovarian folliclular developmental stage and antral follicle size. Sections were also subjected to immunohistochemical (IHC) staining to assess ovarian functional activities via the expression of follicullotropin stimulating hormone receptor (FSHR), Inhibin α, Inhibin β and, Antimullerian hormone (AMH).

Statistical Analysis

All statistical analyses were conducted using SAS 9.4 (SAS Institute Inc., Cary, N.C., USA) assuming an overall significance level of 0.05. Bonferroni's method for multiple comparisons was used to adjust for multiple comparisons where appropriate.

### Results

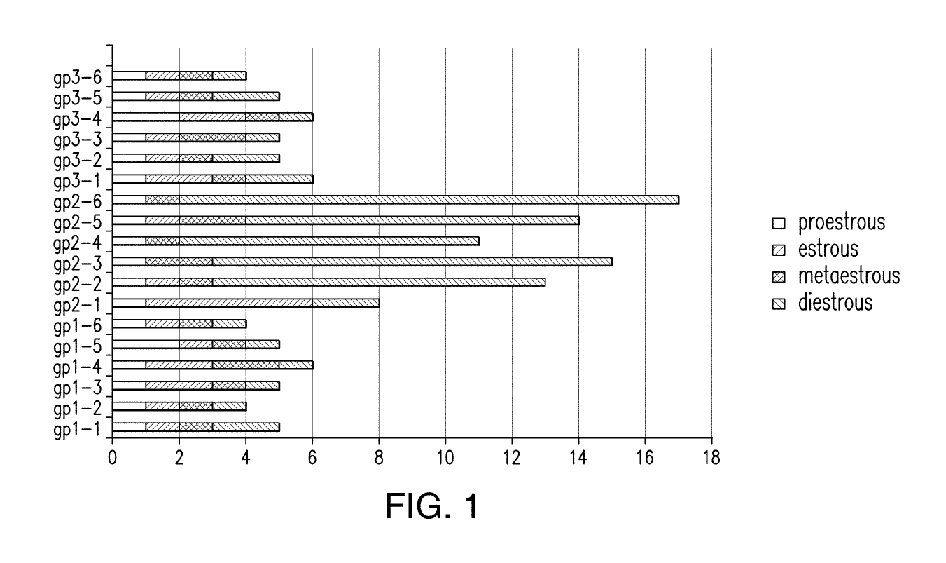

Mice treated with CTX sustained an increase in the length of estrous cycle from the physiological four day cycle observed among control mice to 18-21 day cycle. The estrous cycle among CTX-treated mice was almost totally arrested at diestrus phase although arrested cyclicity was noticed in other stages as well such as proestrus, estrus and metasterus (FIG. 1). A week after intra-ovarian implantation of BMSCs, treated animals started to show changes in vaginal smears though the changes were not as regular as normal animals (i.e., longer or shorter than normal estrus cycle). The mean number of diestrus days or total cycle days did not differ significantly between groups 1 & 3 (p-value=0.6252, 0.7998, respectively). On the other hand, Groups 1 and 3, differed significantly in the mean number of days in the diestrus cycle and the mean number of days for the total cycle when compared to group 2 (FIG. 1, p-value <0.0001).

### Example 2: Total Body Weight and Organ Weight Changes

Results

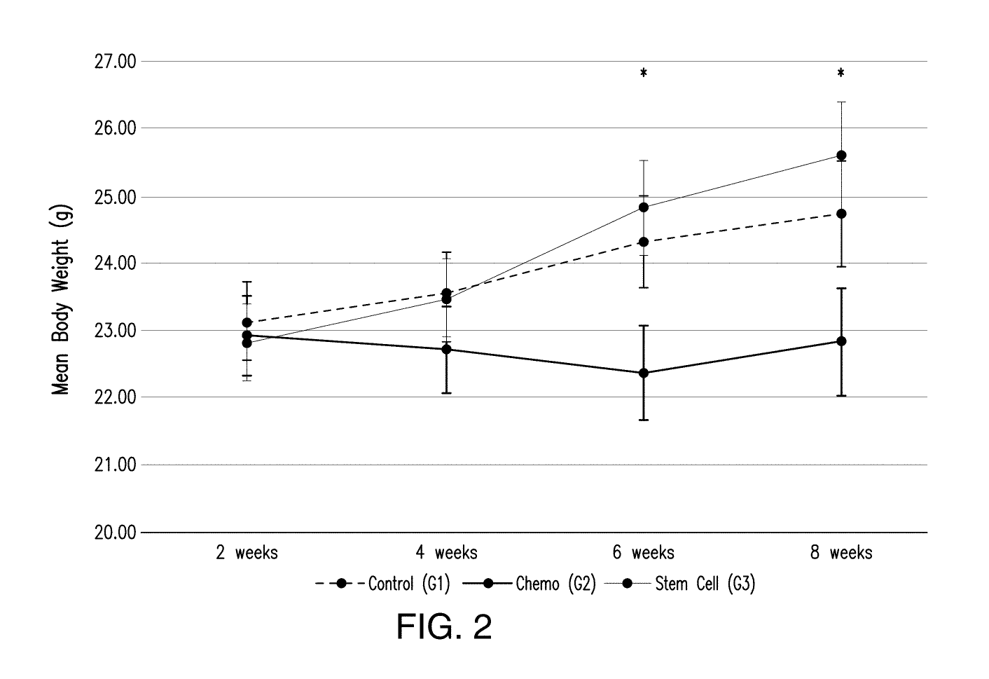

The mean body weight tended to increase with time in groups 1 and 3; whereas, mean body weight remained relatively the same or decreased over time in group 2 as shown in FIG. 2. There was not a significant difference in body weight between groups 1 and 3 at 6 and 8 weeks' time points (p=0.30, 0.13) respectively; however, they had a significantly higher mean body weight than group 2 (p-value <0.0001) (FIG. 2).

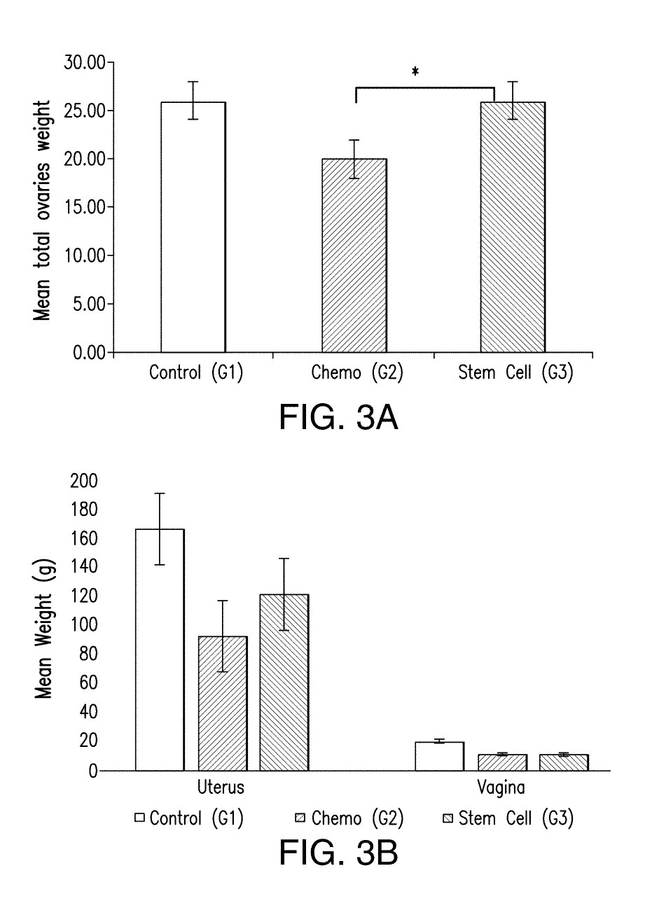

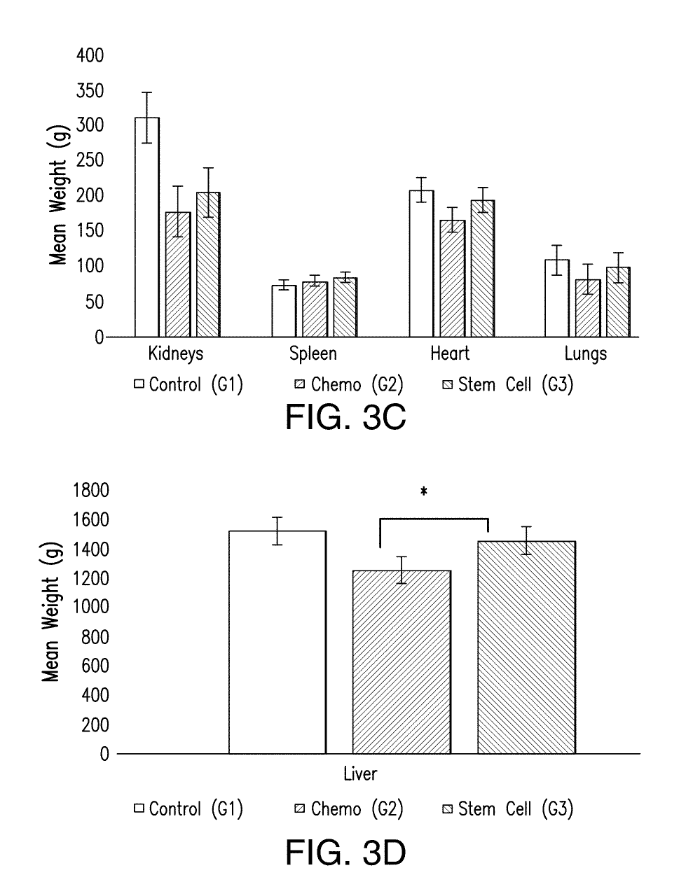

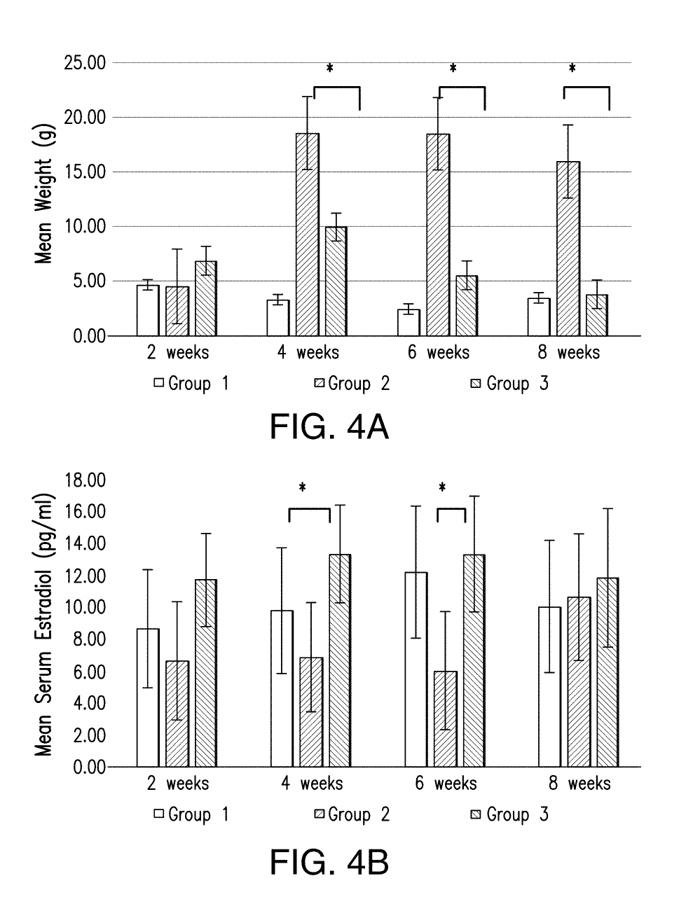

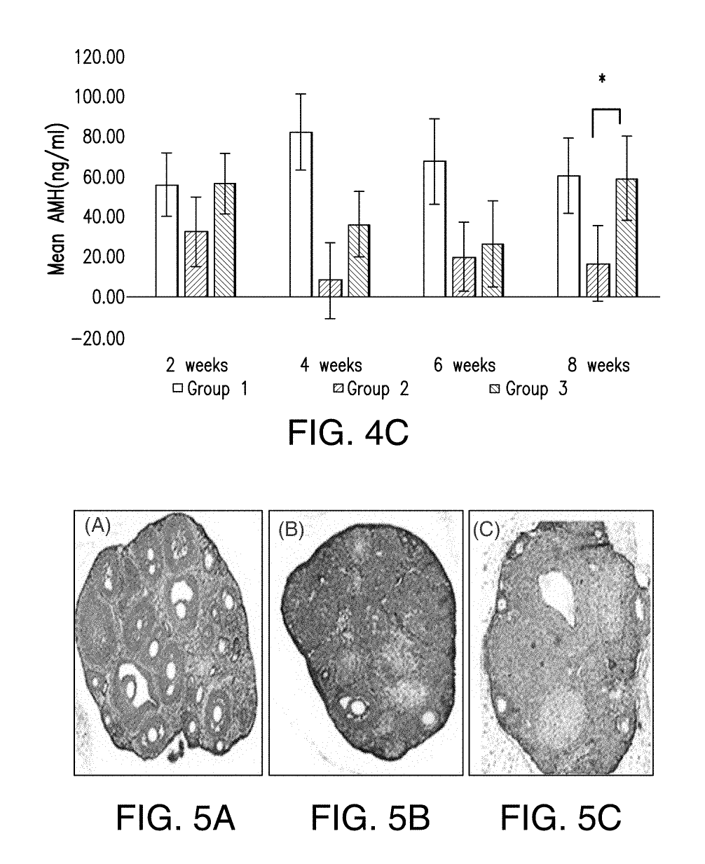

Estrogen responsive organs, demonstrated remarkable increases in weight at all timepoints of the experiment, as shown in FIG. 3. Statistical analysis indicated that there were significant differences among the groups in the mean weight for the ovaries (p=0.0030), uterus (p=0.0015), kidneys (p=0.0001), and liver (p=0.0018), while there were no significant differences among the groups in the mean weight of spleen (p=0.1106), vagina (P=0.66) or lungs (p=0.1778). (FIGS. 3a, b, c, and d)

There were no significant differences in ovary weight between groups 1 and 3 but they both had significantly higher mean total ovary weight than group 2 (P<0.003). These changes are consistent with increased estrogen serum levels and higher estrogen availability in various tissues and organs in BMSC-treated animals.

### Example 3: Hormonal Changes

### Materials and Methods

Blood Collection for Hormonal Assays:

Blood samples from all groups were collected using the retro-orbital technique with sample size averaging 200 ul/animal. Samples were placed on ice until centrifugation at 4° C. at 1500×g for 10 minutes. Sera were harvested and stored frozen at −80° C. until analyzed for ovarian hormonal assay profile (E2 estradiol, P4 progesterone, AMR anitmullerian hormone, FSH follicule-stimulating hormone and luteinizing hormone (LH).

### Results

Blood samples were collected from all animals at baseline and at varying timepoints 2, 4, 6, and 8 weeks from day of surgery. Serum FSH, Antimullerian hormone AMR and Estradiol levels were measured by “The University of Virginia's Center for Research in Reproduction Ligand Assay and Analysis Core. For the detection of Estradiol in mouse serum, an ELISA analysis (Rodent Estradiol ELISA; CalBiotech, Spring Valley, Calif.) was conducted. Assay precision was 6.1% (intra-assay) and 8.9% (inter-assay). Functional sensitivity=3.0 pg/ml. Mouse FSH RIA (MFSH): FSH was assayed by RIA using reagents provided by Dr. A. F. Parlow and the National Hormone and Peptide Program, as previously described. Gay V L, Midgley A R, Jr., Niswender G D. Patterns of gonadotrophin secretion associated with ovulation. Federation proceedings. 1970; 29(6):1880-7. Assay precision was 6.9% (intra-assay) and 9.4% (inter-assay). Functional sensitivity=3.0 ng/ml. Mouse serum samples were measured for AMR using a commercial ELISA kit (Rat/Mouse AMH ELISA, Ansh Labs; Webster, Tex.). Assay precision was 3.6% (intra-assay) and 8.5% (inter-assay). Functional sensitivity=0.28 ng/ml.

### Example 4: Breeding Experiment

Acclimatized mice (N=6/group) were randomly assigned to a control group (group1″), sham chemotherapy group (group 2″) and treatment (group 3″) as aforementioned; however, in this experiment, animal mating was initiated one week after surgical recovery by cohabitating them with age and strain matched breeder males at a ratio of 2:1 (2 control mice, CTX, or treated C57BL6 female to 1 C57BL6 male). Successful mating was determined by the presence of plugs in the vaginal os of females. All resulting pups were collected, counted and examined closely for weight as well as any visible congenital anomalies or abnormal physical findings.

### Example 5: Ovarian Histological Studies

Animals were sacrificed (CO₂ asphyxiation in accordance with GRU animal facility protocols) at 2, 4, 6 and 8 week timepoints after surgery. Organ samples (lung, heart, liver, spleen, ovaries, uterus, vagina and cervix) were collected, weighed and stored at −80° C. until further processing. All organs were weighed by a blinded lab technician, i.e., unaware of group assignment. Mean organ weight was used for comparisons. Tissues were fixed in 10% formalin overnight and embedded in paraffin. Five-micrometer-thick sections were stained with hematoxylin and eosin for light microscopic histological examination. In all samples, the fifth cut was chosen to count the number of follicles and to evaluate follicular development as previously described. Ghadami M, El-Demerdash E, Salama S A, Binhazim A A, Archibong A E, Chen X, et al. Toward gene therapy of premature ovarian failure: intraovarian injection of adenovirus expressing human FSH receptor restores folliculogenesis in FSHR(−/−) FORKO mice. Molecular human reproduction. 2010; 16(4):241-50. To evaluate follicular growth, follicles were classified as preantral, if they contained an oocyte with a visible nucleolus, more than one layer but less than five layers of granulosa cells, and lacked an antral space. Follicles were classified as antral, if they contained an oocyte with a visible nucleolus, more than five layers of granulosa cells, and/or an antral space. Britt K L, Drummond A E, Cox V A, Dyson M, Wreford N G, Jones M E, et al. An agerelated ovarian phenotype in mice with targeted disruption of the Cyp 19 (aromatase) gene. Endocrinology. 2000; 141(7):2614-23.

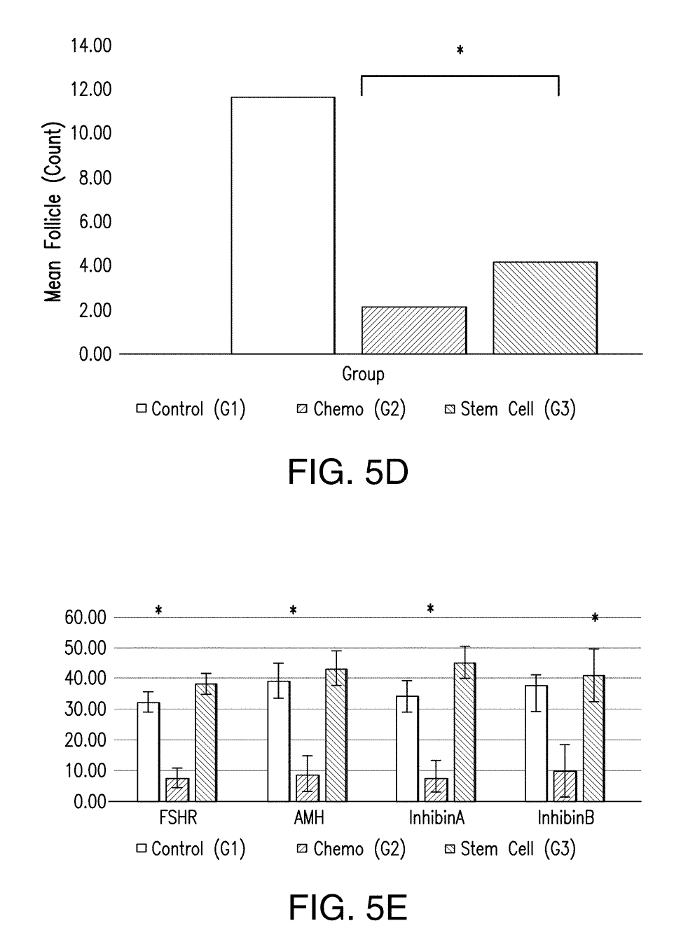

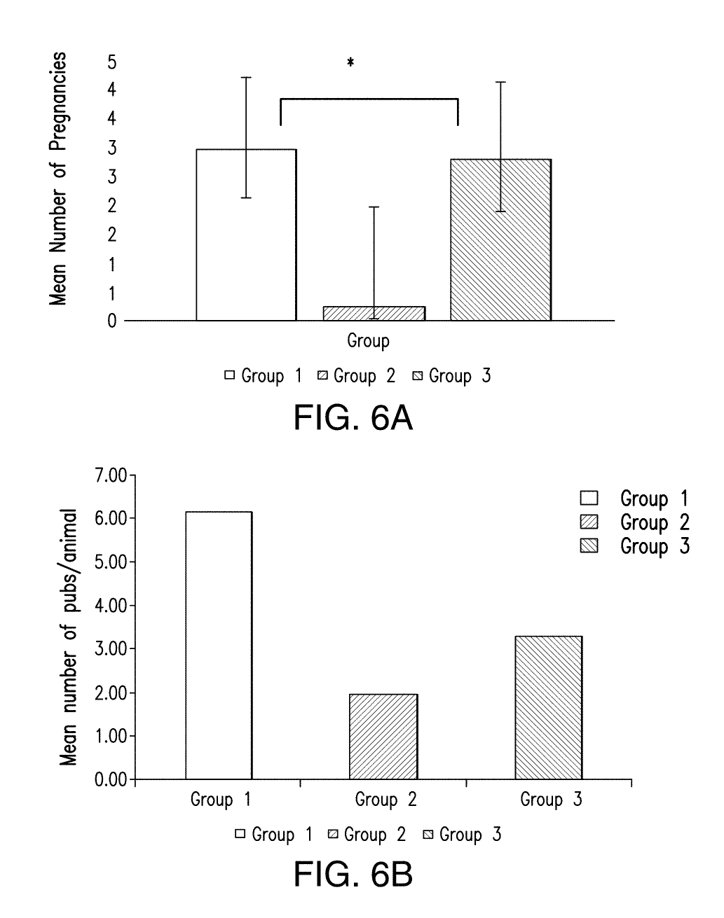

Sections of ovaries (5 μm thick) from controls, CTX and treated mice were subjected to IHC staining in Georgia Regents University's (GRU) IHC Core laboratory. Sections were stained for FSHR, Inhibin α, Inhibin β and AMH to evaluate ovarian functional activities within the three groups. FSHR, a glycoprotein hormone receptor, can serve as a granulosa cell marker and is required for normal ovarian development and follicle maturation in females. AMH is a member of the transforming growth factor β family of growth and differentiation factors. In humans, it is encoded by the AMH gene which is expressed by ovarian granulosa cells in reproductive ages. AMH controls the formation of primary follicles by inhibiting excessive follicular recruitment by FSH. AMH is strongly correlated with follicle pool size. It, therefore, has a role in folliculogenesis and can be used as a marker in determining ovarian age, responsiveness and pathophysiology. Inhibins (α and β) are heterodimeric protein hormones secreted by granulosa cells of the ovary in the female. They selectively suppress the secretion of pituitary follicle stimulating hormone (FSH) and have local paracrine actions in the gonads. Images of ovarian sections were acquired with an Icore Axioplane 2 Nikon TE2000-E inverted microscope, using a 10×, 20× and 40× objective with numerical aperture (NA) 0.30 and 0.75 NA, respectively. Semi-quantitative analysis of mean intensities of controls, CTX and the treatment group for FSHR, Inhibin α, Inhibin β and AMH stained sections were performed using NIS Elements Advanced Research Software. 4 to 6 regions of interest (ROI) were outlined on images obtained from each group using NIS Elements software. Background intensity was subtracted from the ROI, and an intensity threshold was set and kept constant for all images analyzed. Mean intensity per square micrometer area was calculated by dividing the mean intensity units by the area of outlined regions. These results are presented as bar graphs. (FIG. 5b2)

### Example 6: Tracking BMSCs by Immunohistochemical Staining

Anti-Vimentin stained human cells in mouse ovarian tissue (Abcam catalogue number ab8069). Cells were fixed with 4% formaldehyde (10 min), permeabilized with 0.1% Triton X-100 for 5 minutes and then blocked with 1% BSA/10% normal goat serum/0.3M glycine in 0.1% PBS-Tween for 1 hour. Cells were then incubated overnight at +4° C. with ab8069 at 1/100 dilution. Nuclear DNA was labelled with DAPI (shown in blue) and an Image was taken using a confocal microscope (Leica-Microsystems, TCS SP8).

### Example 7: Intra-Ovarian Injected BMSCs were Able to Restore Ovarian Hormone Production, and Reactivate Folliculogenesis in a Chemotherapy-Induced Ovarian Failure Mouse Model

Post BMSCs implantation, group 3 showed detectable estrogenic changes in vaginal smears as early as 1 week. A trend of increased total body weight as well as ovarian weight and other estrogen-responsive organs (uterus & liver) were observed in group 3 as early as 2 weeks post stem cell therapy which became significantly different from group 2 by week 6, post cell implantation. Hematoxylin and Eosin evaluation of the ovaries demonstrated a higher mean follicle count in group 3 compared to group 2. At 4 weeks post treatment, group 3 exhibited a serum hormonal profile of revived ovarian function compared to group 2 including lower follicle stimulating hormone serum (FSH) levels; (p=0.03), and higher Antimullerian hormone serum (AMH) levels (p=0.0005) than group 2. IHC analysis demonstrated higher expression of AMH, FSH receptor, Inhibinα and Inhibinβ in growing follicles in group 3 versus group 2. Human cell-specific tracking studies demonstrated that BMSCs have evenly repopulated growing follicles in treated ovaries. Importantly, breeding data showed significant increases in the number of pregnancies over a 12 week period in group 3 as compared to group 2 (p=0.02). Mice in group 2 had only one pregnancy resulting in the live birth of 2 pups within the 12 week period post cell implantation, while mice in group 3 had a total of 12 pregnancies during the same time period resulting in the live births of 33 pups. All pups in group 3 appeared normal with no visible malformations.

The data show that intra-ovarian injected BMSCs were able to restore ovarian hormone production, and reactivate folliculogenesis in a chemotherapy-induced ovarian failure mouse model resulting in reversed infertility in this preclinical model.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

All references cited herein are incorporated by reference in their entirety. The present invention may be embodied in other specific forms without departing from the spirit or essential attributes thereof and, accordingly, reference should be made to the appended claims, rather than to the foregoing specification, as indicating the scope of the invention.

I claim:

1. A method for treating ovarian failure in a subject in need thereof comprising:
administering an effective amount of bone marrow derived stem cells into an ovary of the subject to restore ovarian hormone production and promote folliculogenesis.

2. The method of claim 1, wherein the ovarian failure is selected from the group consisting of idiopathic premature ovarian failure and chemotherapy-induced ovarian failure.

3. The method of claim 1, wherein the bone marrow derived stem cells have the following surface marker profile: CD105 positive, CD166 positive, CD90 positive, and CD73 positive as well as CD14 negative, CD34 negative, CD45 negative, HLA-DR negative, and CD 19 negative.

4. The method of claim 1, wherein the bone marrow derived stem cells are autologous stem cells.

5. The method of claim 1, wherein the bone marrow derived stem cells are expanded in cell culture prior to administration to the subject.

6. A method for treating chemotherapy-induced ovarian failure in a subject in need thereof comprising:
administering locally to an ovary of the subject an effective amount of stem cells to restore ovarian hormone production and promote folliculogenesis.

7. The method of claim 6, wherein the stem cells are selected from the group consisting of embryonic stem cells, adult stem cells, induced pluripotent stem cells, umbilical cord blood cells, placental blood cells, hematopoietic stem cells, and combinations thereof.

8. The method of claim 6, wherein the stem cells are CD105 positive, CD166 positive, CD90 positive, and CD73 positive as well as CD14 negative, CD34 negative, CD45 negative, HLA-DR negative, and CD 19 negative.

9. A method for treating ovarian failure in a subject in need thereof comprising:
administering platelet enriched plasma directly into an ovary of the subject in an amount effective to restore ovarian hormone production and promote folliculogenesis.

10. A method for treating ovarian failure in a subject in need thereof comprising:
locally administering to an ovary of the subject an effective amount of conditioned cell culture medium harvested from cultured stem cells, wherein the cultured stem cells are CD105 positive, CD166 positive, CD90 positive, and CD73 positive as well as CD14 negative, CD34 negative, CD45 negative, HLA-DR negative, and CD 19 negative.
